(19) United States
(12) Patent Application Publication
(10) Pub. No.: US 2012/0263685 A1
(43) Pub. Date: Oct. 18, 2012

(54) ANTI-TUMOR COMPOSITION COMPRISING HUMAN-DERIVED ADULT STEM CELLS

(75) Inventors: Jeong Chan RA, Gyeonggi-do (KR); Sung Keun KANG, Seoul (KR); Sang Kyu WOO, Gyeonggi-do (KR); Hwa Young YOUN, Seoul (KR); Hee Woo LEE, Seoul (KR); Kyoung Won SEO, Incheon (KR)

(73) Assignee: RNL BIO CO., LTD, Seoul (KR)

(21) Appl. No.: 13/500,764

(22) PCT Filed: Apr. 7, 2010
(86) PCT No.: PCT/KR2010/002139

§ 371 Date: Jun. 19, 2012

(30) Foreign Application Priority Data
Oct. 8, 2009 (KR) 10-2009-0095579

(51) Int. Cl. A61K 35/12; A61K 38/18; C12N 5/0775; A61P 35/02; A61P 35/00; A61K 38/39; A61K 38/19; C12N 5/074; C12N 5/0789

(52) U.S. Cl. 424/93.7; 435/366; 514/7.6; 435/372; 514/8.9; 514/9.1; 514/8.1; 514/8.4; 514/8.5; 514/9.2; 514/9.5; 514/17.2; 514/19.3; 514/19.4; 514/19.6; 514/9.3

(57) ABSTRACT

The present invention relates to a composition for preventing or treating cancer, which contains one or more selected from the group consisting of human adult stem cells and their secretory products, and to a method of preventing or treating cancer using the same. Particularly, the invention relates to the use of adult stem cells that exhibit the effect of preventing or treating cancer by activating the immune system. The human adult stem cells of the invention are administered by a simple method such as intravenous injection and are highly valuable as a cell therapeutic agent for treating various cancer (tumor) diseases. Thus, the adult stem cells will be highly useful in anticancer studies.

### TECHNICAL FIELD

The present invention relates to a composition for preventing or treating cancer, which contains one or more selected from the group consisting of human adult stem cells and their secretory products, and to a method of preventing or treating cancer using the same. More specifically, the present invention relates to novel uses of human adult stem cells and their secretory products for inhibiting the expression of chemokines in cancer (tumor) cells and for activating the immune system.

### BACKGROUND ART

Stem cells refer to cells having not only self-replicating ability but also the ability to differentiate into at least two types of cells, and can be classified into totipotent stem cells, pluripotent stem cells, and multipotent stem cells.

Totipotent stem cells are cells having totipotent properties capable of developing into one perfect individual, and these properties are possessed by cells up to the 8-cell stage after the fertilization of an oocyte and a sperm. When these cells are isolated and transplanted into the uterus, they can develop into one perfect individual. Pluripotent stem cells, which are cells capable of developing into various cells and tissues derived from the ectodermal, mesodermal and endodermal layers, are derived from an inner cell mass located inside of blastocysts generated 4-5 days after fertilization. These cells are called “embryonic stem cells” and can differentiate into various other tissue cells but not form new living organisms. Multipotent stem cells, which are stem cells capable of differentiating into only cells specific to tissues and organs containing these cells, are involved not only in the growth and development of various tissues and organs in the fetal, neonatal and adult periods but also in the maintenance of homeostasis of adult tissue and the function of inducing regeneration upon tissue damage. Tissue-specific multipotent cells are collectively called “adult stem cells”.

Adult stem cells are obtained by taking cells from various human organs and developing the cells into stem cells and are characterized in that they differentiate into only specific tissues. However, recently, experiments for differentiating adult stem cells into various tissues, including liver cells, were dramatically successful, which comes into spotlight.

Meanwhile, cancer is a group of diseases characterized by uncontrolled cell growth, and this abnormal cell growth leads to the formation of a cell mass called a tumor. The cell mass invades the surrounding tissue, and in severe cases, may metastasize to other organs of the body. Cancer is academically called neoplasia.

Methods for treating cancer include surgery, radiation therapy, chemotherapy in which anticancer agents are administered, and the like. Anticancer agents are drugs that act on cancer cells to suppress the proliferation and growth of the cancer cells. Alkylating anticancer agents such as cisplatin or cyclophosphamide, which are currently widely used, exhibit anticancer activity by forming a covalent bond with nitrogen in the nucleotide of DNA. In addition, 5-fluorouracil exhibits anticancer activity either by suppressing enzymes involved in biosynthesis of nucleic acids or by being inserted directly into DNA or RNA. Moreover, antibiotics such as adriamycin exhibit anticancer effects by potentially inhibiting the intrinsic functions of DNA. However, these anticancer agents entail the problem of causing various side effects, including renal poisoning, vomiting and neurotoxicity, because they act not only on tumor cells, but also normal cells, particularly bone marrow cells or intestinal epithelial cells, which rapidly proliferate and differentiate. For this reason, immunotherapy that uses the immune system to fight cancer has received attention as a new method for treating cancer.

Patent documents relating to anticancer therapy which uses stem cells include U.S. Pat. No. 6,210,668 which describes transplanting stem cells to promote the activation of T cells, U.S. Pat. No. 5,843,435 which describes the transplantation of autologous stem cells, U.S. Pat. No. 6,143,292 which describes the transplantation of allogeneic stem cells, and U.S. Patent Publication No. 2008-0241115 relating to an anticancer composition containing mesenchymal stem cells which express a suicide gene. However, there has been no report on the cancer preventive or therapeutic effect of immune system activation caused by intravenously administering adult stem cells.

Accordingly, the present inventors have observed that intravenous administration of adipose mesenchymal stem cells to mice suffering from melanoma led to a significant decrease in the volume of the tumor cells and that treatment of immune cells, extracted from the blood of a cancer patient, with adipose mesenchymal stem cells, led to a significant increase in the proliferation rate of the immune cells, indicating that the adult mesenchymal stem cells have the effect of preventing or treating cancer by activating the immune system, thereby completing the present invention.

### DISCLOSURE OF INVENTION

### Technical Problem

It is an object of the present invention to provide a composition for preventing or treating cancer diseases, the composition containing as an active ingredient one or more selected from the group consisting of human adult stem cells and their secretory products.

Another object of the present invention is to provide a composition for activating the immune system, the composition containing as an active ingredient one or more selected from the group consisting of human adult stem cells and their secretory products.

Still another object of the present invention is to provide a method for preventing or treating cancer, the method comprising a step of administering a composition containing as an active ingredient one or more selected from the group consisting of human adult stem cells and their secretory products.

Yet another object of the present invention is to provide a method for activating the immune system, comprising a step of administering a composition containing as an active ingredient one or more selected from the group consisting of human adult stem cells and their secretory products.

### Technical Solution

To achieve the above objects, the present invention provides a composition for preventing or treating cancer and a composition for activating the immune system, each of the compositions containing as an active ingredient one or more selected from the group consisting of human adult stem cells and their secretory products.

### BRIEF DESCRIPTION OF THE DRAWINGS

FIG. 1 is a process diagram showing the test method of Example 3.

FIG. 2 is a graphic diagram showing the results of Example 3, obtained by measuring the change in tumor volume as a function of time (days) for a test group and a control group.

FIG. 3 is a graphic diagram showing the results of Example 3, obtained by measuring survival rate as a function of time (days) for a test group and a control group.

FIG. 4 is a process diagram showing the test method of Example 4.

FIG. 5 is a graphic diagram showing the results of Example 4, obtained by measuring the change in tumor volume as a function of time (days) for a test group and a control group.

FIG. 6 is a graphic diagram showing the results of Example 4, obtained by measuring survival rate as a function of time (days) for a test group and a control group.

FIG. 7 is a graphic diagram showing the results of Example 5, which show the survival rate of stem cells in a test group and control group 1, obtained by measuring the number of viable stem cells using a WST-1 assay.

FIG. 8 is a graphic diagram showing the results of Example 5, which show the survival rate of stem cells in a test group and control group 1, obtained by measuring absorbance using a WST-1 assay.

FIG. 9 is a graphic diagram showing the results of a WST-1 assay in Example 5, which show the survival rates of stem cells in a test group and control group 2, obtained by measuring the number of viable stem cells.

FIG. 10 is a graphic diagram showing the results of a WST-1 assay in Example 5, which show the survival rates of stem cells in a test group and control group 2, obtained by measuring absorbance.

FIG. 11 shows the results of Example 6 and is a set of confocal micrographs for a test group, control group 1 and control group 2.

FIG. 12 is a graphic diagram showing the results of Example 6, which show a difference in the number of stem cells between a test group and control group 1 at various points of time.

FIG. 13 is a graphic diagram showing the results of Example 6, which show a difference in the number of stem cells between a test group and control group 2 at various points of time.

### BEST MODE FOR CARRYING OUT THE INVENTION

Hereinafter, the present invention will be described in detail.

Stem cells refer to cells having not only self-replicating ability but also the ability to differentiate into at least two types of cells.

Adult stem cells are stem cells that appear either in the stage in which each organ of an embryo is formed after the developmental process or in the adult stage. It is known that adult stem cells are multipotent and capable of differentiating into tissue- and organ-specific cells. Such multipotent stem cells, which are stem cells capable of differentiating into cells specific to tissues and organs containing these cells, are involved not only in the growth and development of various tissues and organs in the fetal, neonatal and adult periods but also in the maintenance of homeostasis of adult tissue and the function of inducing regeneration upon tissue damage.

In the present invention, adult stem cells, preferably human adult stem cells, are used.

These adult stem cells may be derived from tissues, including adipose, uterus, marrow, muscle, placenta, umbilical cord blood, or skin (epithelium). Preferably, adult stem cells derived from adipose tissue or epithelial tissue such as a hair follicle or an amnion may be used. Particularly, mesenchymal stem cells (MSCs) are preferably used. Most preferably, human adipose tissue-derived mesenchymal stem cells (AT-MSCs) may be used.

As used herein, “adipose tissue-derived adult stem cells” or “adipose tissue-derived mesenchymal stem cells” are undifferentiated adult stem cells isolated from adipose tissue and are also referred to herein as “adipose stem cells”. These cells can be obtained according to any conventional method known in the art.

The adipose stem cells can be obtained using any conventional medium known in the art to be suitable for stem cell culture. Preferably, DMEM (Dulbecco's modified Eagle medium) or Keratinocyte-SFM (Keratinocyte serum-free medium) is used in the present invention.

The medium for culturing adipose stem cells may be supplemented with additives that promote the proliferation of the undifferentiated phenotype of the adipose stem cells while inhibiting the differentiation of the cells. Also, the medium may generally contain a neutral buffer (such as phosphate and/or high concentration bicarbonate) in isotonic solution; a protein nutrient (e.g., serum such as FBS, serum replacement, albumin, or essential and non-essential amino acids such as glutamine). Furthermore, it may contain lipids (fatty acids, cholesterol, an HDL or LDL extract of serum) and other ingredients found in most stock media of this kind (such as insulin or transferrin, nucleosides or nucleotides, pyruvate, a sugar source such as glucose, selenium in any ionized form or salt, a glucocorticoid such as hydrocortisone and/or a reducing agent such as β-mercaptoethanol).

Also, with a view to protecting cells from adhering to each other, adhering to a vessel wall, or forming clusters that are too big, the medium may be beneficial to include an anti-clumping agent, such as those sold by Invitrogen (Cat # 0010057AE).

Among them, one or more of the following additional additives may advantageously be used:
stem cell factor (SCF, Steel factor), other ligands or antibodies that dimerize c-kit, and other activators of the same signal transduction pathway
ligands for other tyrosine kinase related receptors, such as the receptor for platelet-derived growth factor (PDGF), macrophage colony-stimulating factor, Flt-3 ligand and vascular endothelial growth factor (VEGF)
factors that elevate cyclic AMP levels, such as forskolin
factors that induce gp130, such as LIF or Oncostatin-M
hematopoietic growth factors, such as thrombopoietin (TPO)
transforming growth factors, such as TGFβ1
other growth factors, such as epidermal growth factor (EGF)
neurotrophins, such as CNTF
N-acetyl-L-cysteine (NAC)
hydrocortisone
ascrobic acid

Particularly, a medium which is used to culture adipose stem cells in one embodiment of the present invention preferably contains NAC, ascrobic acid, calcium, insulin and hydrocortisone. More preferably, it contains FBS, NAC, ascrobic acid, calcium, rEGF, insulin and hydrocortisone.

In addition, the composition of the present invention may contain as an active ingredient the secretory products of the human adult stem cells. These secretory products include various cytokines, amino acids and growth factors, for example, TGF, bFGF, IGF, KGF, HGF, fibronectin, VEGF or procollagen.

Particularly, in the present invention, proteins secreted from adipose stem cells were analyzed, and as a result, it was seen that TGF (166.7 pg/ml), bFGF (916.15 pg/ml) , IGF (2490 pg/ml) , KGF (1054 pg/ml) , HGF (20778 pg/ml), fibronectin (2402.3 pg/ml), VEGF (6501.72 pg/ml) and procollagen (842.23 ng/ml) were secreted at high concentrations. In addition, as the number of passages of adipose stem cells increased, VEGF, a protein secreted from the adipose stem cells, showed a tendency to increase from 434.62 pg/ml to 940.77 pg/ml, but HGF (average concentration: 391.0 pg/ml), TGF (average concentration: 552.7 pg/ml), IGF (average concentration: 207.5 pg/ml) and procollagen (average concentration: 556.6 ng/ml) did not significantly change depending on the number of passages.

Meanwhile, these secretory products of human adult stem cells inhibit the expression of chemokines in tumor cells or enhance the function of immune cells, thereby greatly contributing to the activation of the immune system.

In addition, the human adult stem cells and/or their secretory products may be used as a broth of human adipose tissue-derived adult stem cells, obtained by culturing human adipose tissue-derived adult stem cells in a medium containing certain components, harvesting the culture medium and removing cell debris from the culture medium. Alternatively, extracts from the culture broth may be used alone or in combination.

In other words, the composition of the present invention may be a composition containing adult stem cells and their secretory products and medium components, or a composition containing the cell secretory products and the medium components, or a composition containing the cell secretory products alone or in combination with adult stem cells, or a composition containing only adult stem cells that produce secretory products in vivo.

In one aspect, the present invention is directed to the use of human adult stem cells for the treatment of cancer (tumor) diseases. Therefore, the present invention is directed to a composition for preventing or treating cancer diseases, wherein the composition contains human adult stem cells and their secretory products as active ingredients, and to a method of treating cancer diseases using the same.

Meanwhile, cancer is a group of diseases characterized by uncontrolled cell growth, and this abnormal cell growth leads to the formation of a cell mass called a tumor. The cell mass invades the surrounding tissue, and in severe cases, may metastasize to other organs of the body.

As used herein, the term “anticancer” is meant to include not only treating cancer diseases, i.e., inhibiting the proliferation of cancer cells or killing cancer cells, but also preventing cancer diseases, i.e., increasing resistance to cancer before the onset of cancer. Thus, the term “preventing or treating cancer” is used herein interchangeably with the term “anticancer”.

Unless otherwise indicated, the term “treating”, as used herein, means reversing, alleviating, inhibiting the progress of, or preventing the disorder or condition to which such term applies, or one or more symptoms of such disorder or condition. The term “treatment”, as used herein, refers to the act of treating as “treating” is defined immediately above. Thus, as used herein, “treating” or “treatment” of a cancer in a mammal includes one or more of: (1) inhibiting growth of the cancer, i.e., arresting its development, (2) preventing spread of the cancer, i.e., preventing metastases, (3) relieving the cancer, i.e., causing regression of the cancer, (4) preventing recurrence of the cancer, and (5) palliating symptoms of the cancer.

Cancer diseases which can be treated by the inventive composition for preventing or treating cancer include glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, meduliary carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcoma, chondrosarcoma, osteogenic sarcoma, and leukemia.

Such cancer diseases are chronic intractable diseases which are, in many cases, not basically treated by surgery, radiotherapy and chemotherapy, give pain to patients, and ultimately lead to death. Surgical therapy, chemotherapy (treatment with anticancer agents), radiotherapy and the like have been much developed in the last several decades, but are not the ultimate solution to treat cancer.

Therefore, immunotherapy is now expected as a new cancer treatment, which uses immunity of our body. Immunotherapy is indirect treatment that treats cancer by activating patient's immune response, whereas surgery, chemotherapy and radiotherapy directly attack cancer cells among cancer treatments.

Examples of immunotherapy against cancer include a treatment method in which an immune booster is administered such that immune cells attack cancer cells, and a method in which important immune cells (lymphocytes) that provide cell-mediated immunity are used directly as an agent for treating cancer. Immunotherapy uses an immune response in the human body, and thus can minimize side effects which cannot be avoided in existing cancer therapies, and can cure cancer without causing pain. In this sense, immunotherapy can be considered the safest therapy.

The present invention is directed to preventing or treating cancer (i.e., providing anticancer activity) by using adult stem cells to activate the immune system.

In another aspect, the present invention is directed to the use of human adult stem cells for the activation of the immune system. Therefore, the present invention is directed to a composition for activating the immune system, wherein the composition contains human adult stem cells and their secretory products as active ingredients, and to a method of activating the immune system using the same.

As used herein, the term “activating the immune system” means increasing the proliferation rate and activity of immune cells in vivo to enhance immunity. Major immune cells that provide immunity against cancer in vivo can be classified into three types: macrophages, B lymphocytes (B cells), and T lymphocytes (T cells).

Among T lymphocytes, particularly natural killer T lymphocytes (NKT cells) combine T cell receptor with NK cell receptor and have the characteristics of separate lymphocytes that are T cells and NK cells. The NKT cells play an important role in inhibiting the metastasis of cancers cells to the liver and lung in mice and in eliminating cancer cells, parasites, and infectious bacteria such as Listeria and tuberculous bacilli, from cells. Also, cells that can effectively arrest cancer cells include, in addition to NKT cells, natural killer cells, LAK cells (lymphokine-activated killer cells and macrophages. In other words, it was demonstrated that the activities of natural killer cells, LAK cells and macrophages are effective in arresting the growth of cancer cells and the metastasis of cancer and that the activation of natural killer cells by immune stimulants effectively inhibits the metastasis and spread of cancer cells.

In addition, the anticancer, anti-metastasis and antiviral activities caused by activation of the immune system are mediated by secretion of various cytokines. Typical cytokines known to be associated with anticancer, anti-metastasis and antiviral activities include interferon-γ (IFN-γ), tumor-necrosis factor-alpha (TNF-α) and the like. Interferon-γ is produced mainly in T cells and serves to regulate immune responses and activate immune cells, such as T cells, B cells, neutrophils, natural killer cells and macrophages, to attack cancer cells. Thus, interferon-γ is used as an agent for treating chronic myelogenous leukemia, renal cancer and the like. In addition, interferon-γ has a strong activity of inhibiting DNA synthesis and cell proliferation, and thus is used not only as cancer therapeutic agents, but also as clinical agents that inhibit viral infection, multi-drug resistant organism and fungal infection by inhibiting the proliferation of viruses and bacteria, like other interferons. Meanwhile, TNF-α which is produced mainly by macrophages is involved in various immune responses such as inflammatory responses and shows strong toxicity against cells, particularly cancer cells. However, a method of using cytokines directly in anticancer therapy can cause side effects, including unexpected inflammatory responses and vomiting. For this reason, in recent years, a method of treating cells with only a specific cytokine has been avoided, and many attempts have been made to find substances that activate the immune system.

Particularly, the human adult stem cells of the present invention are characterized by secreting specific cytokines that activate the immune system. In addition, some of the secretory products show the effect of treating cancer diseases by inhibiting the expression of chemokines in cancer (tumor) cells.

In order to prevent or treat cancer (tumor) diseases by activating the immune system, the composition of the present invention is administered in a therapeutically effective amount. Also, in order to prevent or treat cancer (tumor) diseases by activating the immune system, the composition of the present invention is preferably administered at the early onset stage of cancer diseases.

used herein, the term “therapeutically effective amount” means that an amount of the compound being administered alleviates to some extent one or more of the symptoms of the disease being treated, Therefore, the therapeutically effective amount refers to an amount that has effect of: (1) reversing the rate of progress of a disease; (2) inhibiting the progress at the disease to some extent; or (3) relieving (preferably eliminating) one or more symptoms associated with the disease to some extent.

The composition therapeutic agent) of the present invention may be clinically administered parenterally, such as intramuscularly or intravenously, or may be administered directly to a disease area. Most preferably, the composition of the present invention is administered by intravenous injection.

For injection, the composition of the present invention may preferably be formulated with a pharmacologically acceptable buffer, such as Hank's solution, Ringer's solution or physiological saline buffer. For transmucous administration, non-invasive agents suitable for a barrier through which the composition is to be passed are used in formulation. Such non-invasive agents are generally known in the art.

Formulations for parenteral administration include sterilized aqueous solutions, non-aqueous solvents, suspending agents, emulsifying agents etc. Suspending agents and emulsifying agents that may be used in the present invention include vegetable oils, such as propylene glycol, polyethylene glycol and olive oil, and injectable ester such as ethyl oleate.

Thus, in another aspect, the present invention is directed to a method for activating the immune system or a method for treating cancer disease, each of the methods comprising intravenously administering a composition containing adipose tissue-derived adult stem cells.

For humans, the cell therapeutic agent of the present invention may conventionally be administered once or several times at a dose of 10⁴-10¹⁰ cells/body, and preferably 10⁶-10⁸ cells/body. Particularly, the composition of the present invention preferably contains adult stem cells at a concentration of 1×10⁵ cells/100 μl to 3×10⁵ cells/100 μl, and most preferably 2×10⁵ cells/100 μl.

However, it should be understood that the actual dose of the active ingredient will be determined in view of various related factors, including the disease to be treated, the route of administration, the patient's age, sex and weight, and the severity of the disease. Thus, the above-described dose is not intended to limit the scope of the present invention in any way.

### EXAMPLES

Hereinafter, the present invention will be described in further detail with reference to examples. It will be obvious to those skilled in the art that these examples are illustrative purposes only and are not to be construed to limit the scope of the present invention.

### Example 1

### Isolation of Mesenchymal Stem Cells from Human Adipose Tissue

Human adipose tissue was isolated from abdominal fat by liposuction and washed with PBS. The isolated tissue was cut finely and then digested in DMEM media supplemented with collagenase type 1 (1 mg/ml) at 37° C. for 2 hours. After washing with PBS, the digested tissue was centrifuged at 1000 rpm for 5 minutes. The supernatant was suctioned off, and the pellets remaining at the bottom were washed with PBS and then centrifuged at 1000 rpm for 5 minutes. The resulting cells were filtered through a 100-gm mesh filter to remove the debris, after which the cells were washed with PBS and then cultured overnight in DMEM (10% FBS, 2 mM NAC, 0.2 mM ascorbic acid).

Then, non-adherent cells were removed by washing with PBS, and the remaining cells were subcultured while the medium was replaced with Keratinocyte-SFM medium (containing 5% FBS, 2 mM NAC, 0.2 mM ascorbic acid, 0.09 mM calcium, 5 ng/ml rEGF, 5 μg/ml insulin and 74 ng/ml hydrocortisone) at 2-day intervals, thereby isolating adipose tissue-derived multipotent mesenchymal stem cells.

The sources of various media and reagents used in the following Examples are shown in Table 1 below.

TABLE 1

| Items | Brands | |
|---|---|---|
| Ascorbic acid | Sigma | USA |
| CaCl₂ | Sigma | USA |
| Collagenase type I | Gibco | USA |
| DMEM (Dulbecco's Modified Eagle's Media) | Gibco | USA |
| DPBS (Dulbecco's Phosphate-Buffered | Welgene | [symbol] |
| Salines) | | |
| EGF (Epidermal growth factor) | Gibco | USA |
| FBS (Fetal Bovine Serum) | Gibco | USA |
| Hydrocortisone | Sigma | USA |
| Insulin | Gibco | USA |
| K-SFM (Keratinocyte-SFM) | Gibco | USA |
| NAC (N-acetyl Cysteine) | Sigma | USA |

Meanwhile, in order to examine the secretory products of the adipose stem cells by analyzing the culture broth of the adipose stem cells, the culture broth was sampled and centrifuged to remove the cell debris, after which the sample was tested immediately or freeze-dried at −20° C. In subsequent experiment procedures, a process of freezing and thawing the sample was avoided if possible.

A reagent, a stock solution for making standard control samples, and a test sample were prepared. In order to make the standard control samples, a stock solution of the protein to be measured was serially diluted in a polypropylene tube, thus making a total of seven concentrations between the highest concentration and the lowest concentration.

Antibody-bound microplate strips were prepared, and the serially diluted standard control sample and the supernatant culture medium of adipose stem cells were dispensed on the microplates and allowed to react with the plates. After completion of the reaction, the standard control sample and the test sample were recovered, and the microplates were washed three times with washing buffer. A conjugate of an antibody against the protein to be measured was added to and allowed to react with each of the microplates. After completion of the reaction, the conjugate was recovered, and the microplates were washed three times with washing buffer. Then, a substrate solution was added to and allowed to react with each of the microplates.

In order to stop the enzymatic reaction of the substrate solution, a stop solution was added to each of the microplates. Within 30 minutes after the addition of the stock solution, the absorbance at a wavelength of 540-570 nm was measured using a spectrophotometer. Further details follow the information of the ELISA kit.

VEGF (R&D system Cat. No. DVE00)

HGF (R&D system Cat. No. DHG00)

TGF-b1 (R&D system Cat. No. DB100B)

IGF-1 (R&D system Cat. No. DG100)

Procollagen (TAKARA Cat. No. MK101)

The analysis of proteins secreted from the adipose stem cells revealed that TGF (166.7 pg/ml), bFGF (916.15 pg/ml), IGF (2490 pg/ml), KGF (1054 pg/ml), HGF (20778 pg/ml), fibronectin (2402.3 pg/ml), VEGF (6501.72 pg/ml) and procollagen (842.23 ng/ml) were secreted at high concentrations.

### Example 2

### Anticancer Effect of Human Adipose Tissue-Derived Mesenchymal Stem Cells (AT-MSCs)

Adipose tissue-derived mesenchymal stem cells (AT-MSCs) obtained in Example 1 were administered into a C57BL/7 mouse melanoma model by intravenous (IV) injection, and then the ability of the stem cells to cure tumor was examined.

Group 1 (control group): 100 μl of saline was administered to the tail vein of four 8-week-old female mice by intravenous (IV) injection three times at 3-day intervals, and 13 days after the first administration, melanoma cells (B16) were injected into the mice by subcutaneous (S.C) injection.

TABLE 2

| | | | | Injection |
|---|---|---|---|---|
| Date | days | types | Amount | method |
| July 7 | −13 | saline | 100 μl | I.V |
| July 10 | −10 | saline | 100 μl | I.V |
| July 13 | −7 | saline | 100 μl | I.V |
| July 20 | 0 | B16 | 100 μl | S.C |

Group 2 (test group; preventive group): the adipose tissue-derived mesenchymal stem cells (AT-MSCs) obtained in Example 1 were administered at a concentration of 2×10⁵ cells/100 μl into the tail vein of three 8-week-old female mice by intravenous (IV) injection three times at 3-day intervals, and 13 days after the first administration, melanoma cells (B16) were injected into the mice by subcutaneous (S.C) injection.

TABLE 3

| | | | | Injection |
|---|---|---|---|---|
| Date | Days | Types | Amount | method |
| July 7 | −13 | AT-MSCs | 2 × 10⁵/100 μl | I.V |
| July 10 | −10 | AT-MSCs | 2 × 10⁵/100 μl | I.V |
| July 13 | −7 | AT-MSCs | 2 × 10⁵/100 μl | I.V |
| July 20 | 0 | B16 | 2 × 10⁵/100 μl | S.C |

15, 18, 21, 23, 25, 28 and 30 days after administration of the cancer cells, the tumor volume was measured.

The results of the measurement are shown in Table 4 below.

TABLE 4

| | Tumor volume (mm³) | | | | | | |
|---|---|---|---|---|---|---|---|
| | | | | | Preventive group | | |
| Day | Control group (four mice) | | | | (three mice) | | |
| 0 | 0 | 0 | 0 | 0 | 0 | 0 | 0 |
| 15 | 743.5521 | 583.7945 | 357.216 | 515.1448 | 0 | 140.14 | 178.7597 |
| 18 | 2837.596 | 2722.149 | 871.0455 | 3436.954 | 1075.67 | 713.7332 | 451.321 |
| 21 | 8230.37 | 7234.08 | 5292.162 | 4761.551 | 1558.082 | 2928.739 | 583.2427 |
| 23 | 14028.13 | 8865.858 | 5281.551 | 9903.105 | 3142.008 | 7249.479 | 1193.715 |
| 25 | 18545.24 | 12908.96 | 6661.44 | 10970.89 | 3883.913 | 10094.21 | 1998.776 |
| 28 | 23727.04 | 17839.29 | 8323.57 | 17646.96 | 4682.703 | 13591.7 | 3102.842 |
| 30 | 25418.28 | 20389.27 | Die | 15171.8 | 14815.8 | 19067.11 | 5306.461 |

As can be seen in Table 4 above, it could be observed that, in the case where the adipose tissue-derived mesenchymal stem cells (AT-MSCs) were administered by intravenous (IV) injection before injection of the cancer cells, the proliferation of the tumor cells was significantly inhibited.

### Example 3

### Cancer Preventive Effect of Human Adipose-Derived Mesenchymal Stem Cells (AT-MSCs)

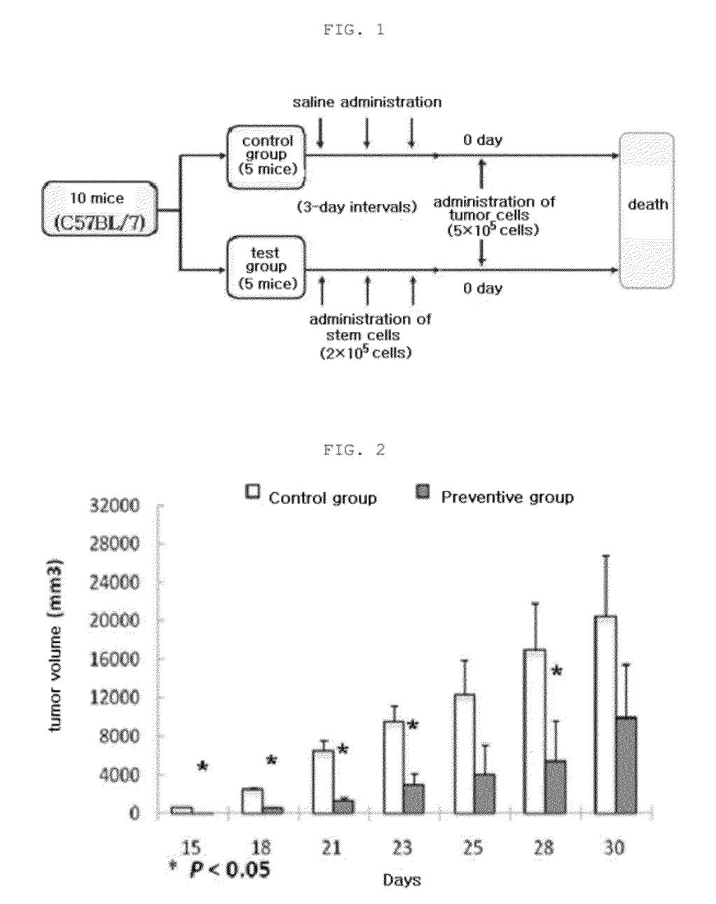

In order to analyze the cancer preventive effect of the adipose tissue-derived mesenchymal stem cells against melanoma, the adipose tissue-derived mesenchymal stem cells were administered into a mouse model, after which melanoma in the mice was induced. In a test group, the adipose tissue-derived mesenchymal stem cells were administered at a concentration of 2×10⁵ cells/100-200 μl into the tail vein of C57BL/7 mice (n=5) 3 times at 3-day intervals, and 7 days after the last administration, B16/F10 melanoma cells were administered subcutaneously into the back of the mice at a concentration of 5×10⁵ cells/100-200 μl. Then, the volume of melanoma and viability were measured. In a control group (n=5), 100-200 μl of saline was administered in place of the adipose tissue-derived mesenchymal stem cells (see FIG. 1).

At 2-day intervals between day 15 and day 30 after administration of the melanoma cells (day 0), the production of melanoma was observed, and behaviors (i.e., mobility and the gloss of hairs) were observed. The volume of melanoma was measured using vernier calipers and calculated according to the equation [(width²*length)/2]. The cell viability was determined by measuring the survival time.

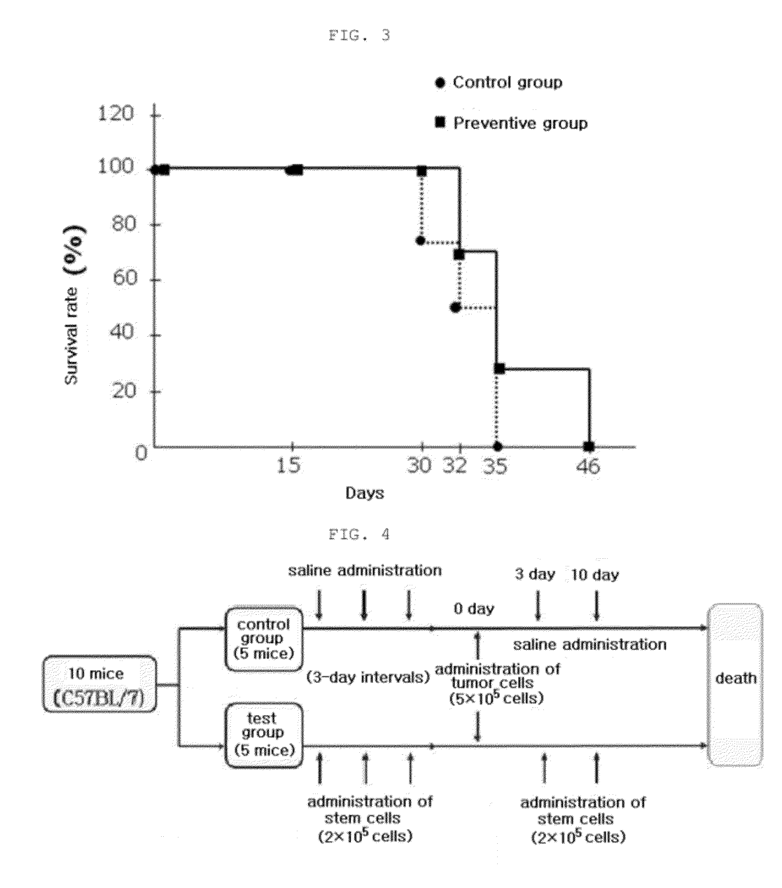

The volume of melanoma (see FIG. 2) and the survival rate of cells were measured between day 15 and day 30 after administration of the melanoma cells (day 0), as a result, it could be seen that, in the test group administered with the adipose tissue-derived mesenchymal stem cells, the formation of the tumor was inhibited and the survival rate of the cells was increased, compared to those in the control group (administered with saline). Such results indicate that the human adipose-derived mesenchymal stem cells have an anticancer effect against melanoma.

### Example 4

### Cancer Preventive and Therapeutic Effects of Human Adipose Tissue-Derived Mesenchymal Stem Cells (AT-MSCs)

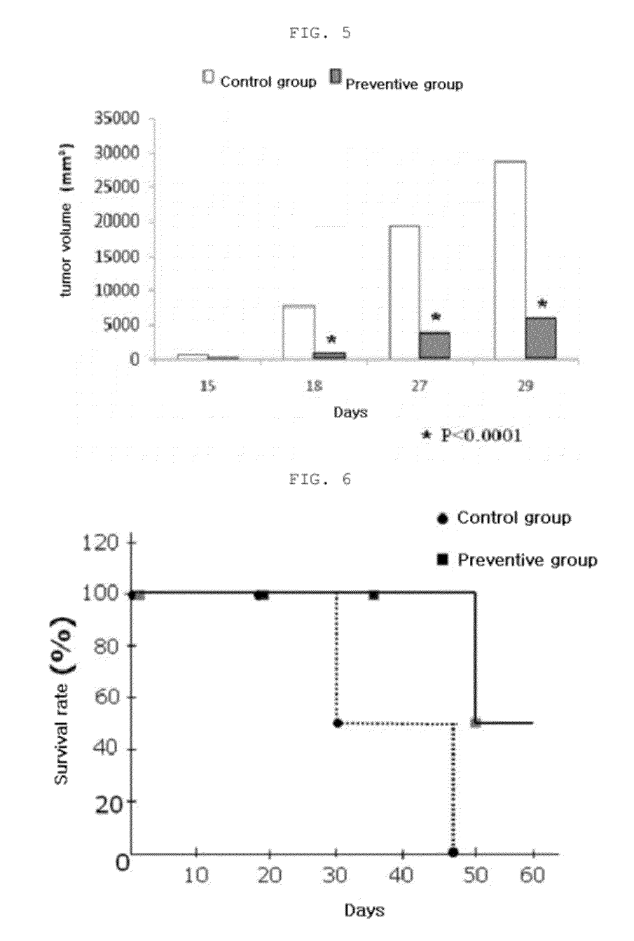

In order to analyze the anticancer effect of administration of the adipose-derived mesenchymal stem cells against melanoma, the adipose-derived mesenchymal stem cells were first administered to a mouse model to induce melanoma, and melanoma was then induced in the mice, after which the adipose-derived mesenchymal stem cells were administered to the mice. Specifically, the experiment was performed in the following manner. In a test group, the adipose-derived mesenchymal stem cells were administered at a concentration of 2×10⁵ cells/100-200 μl into the tail vein of C57BL/7 mice (n=5) three times at 3-day intervals, and 7 days after the last administration, B16/F10 melanoma cells were injected subcutaneously into the back of the mice at a concentration of 5×10⁵ cells/100-200 μl. Then, 3 days and 10 days after administration of the tumor cells (0 day), the adipose-derived mesenchymal stem cells were administered into the tail vein of the mice at a concentration of 2×10⁵ cells/100-200 μl, and the volume of melanoma and the survival rate of cells were measured. In a control group (n=5), 100-200 μl of saline was administered in place of the adipose-derived mesenchymal stem cells (FIG. 4).

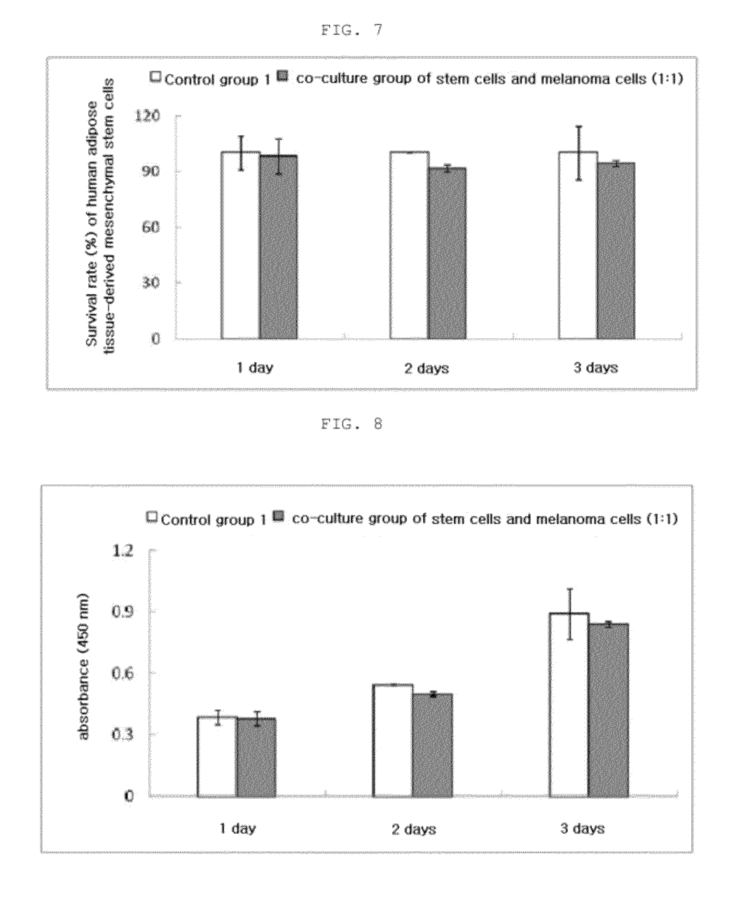

At 2-day intervals between day 15 and day 29 after administration of the melanoma cells, the volume of melanoma (FIG. 5) and the survival rate of cells (FIG. 6) were measured in the same manner as described in Example 3. As a result, it could be seen that, in the test group administered with the adipose-derived stem cells, the tumor formation was significantly inhibited and the cell survival rate was increased, compared to those in the control group (administered with saline). Such results indicate that the human adipose-derived mesenchymal stem cells have cancer preventive and therapeutic effects against melanoma and that the treatment method comprising a step of administering the adipose-derived mesenchymal stem cells to the mouse model, then inducing melanoma in the mice and then administering adipose-derived mesenchymal stem cells again to the mice has a significantly excellent anticancer effect against melanoma compared to the results obtained by administering the adipose-derived mesenchymal stem cells to the mouse model, inducing melanoma in the mice and then measuring the anticancer effect of the cells against melanoma.

### Example 5

### Examination of the Inhibitory Effect of Human Adipose Tissue-Derived Mesenchymal Stem Cells (AT-MSCs) on Proliferation of Cancer Cells by Indirect Co-Culture

It was demonstrated in the in vivo experiments of Examples 2 to 4 that the human adipose tissue-derived mesenchymal stem cells have an anticancer effect against melanoma. In order to verify this fact, the indirect co-culture and direct co-culture of human adipose tissue-derived mesenchymal stem cells and melanoma cells were performed and the inhibitory effect of the human adipose tissue-derived mesenchymal stem cells on the proliferation of the melanoma cells was analyzed in vitro.

In order to perform the indirect co-culture of the cells, in a test group, 5×10⁴ human adipose tissue-derived mesenchymal stem cells were attached to the bottom of each well of a 24-well plate (which can perform co-culture of two cell types), and 5×10⁴ melanoma cells were attached to an insert membrane placed in each well. Then, the cells were cultured an antibiotic-containing OPTI-MEM medium (supplemented with 10% FBS, Gibco) under the conditions of 5% CO₂ and 37° C. for 1, 2 and 3 days. In control group 1, the stem cells (AT-MSCs) alone were cultured under the above conditions, and in control group 2, melanoma cells (B16) alone were cultured under the above conditions.

Then, the proliferative ability of the cells was analyzed by a WST-1 assay. The WST-1 assay is a method of quantifying cell proliferative ability and cell viability by measuring color development. Based on the fact that a tetrazolium salt (WST-1) is converted to a formazan dye by mitochondrial dehydrogenase in viable cells, measurement can be performed by non-RI in place of a [³H]-thymidine binding assay. A tetrazolium salt added to medium is converted to a formazan dye by succinate-tetrazolium-reductase (EC 1.3.99.1) which is present in the mitochondrial breathing chain and has activity only in viable cells. As the number of viable cells increases, the entire activity of mitochondrial dehydrogenase in a sample increases, thus inducing an increase in the production of the formazan dye. Thus, the formazan dye and the number of cells having metabolic activity in medium show a linear correlation. This measurement method has advantages in that there is no need to wash or bind cells and in that procedures ranging from microculture to the analysis of data by an ELISA reader can be performed on the same microtiter plate. In addition, cell proliferative ability and cell viability can be measured with a spectrophotometer by non-RI in a drug sensitive measurement method.

In the WST-1 assay of this Example, the well and insert membrane having the cells attached thereto were isolated, and 0.5 ml of OPTI-MEM medium (supplemented with 10% FBS, Gibco) and 50 μl of a WST-1 reagent solution containing a tetrazolium salt were added thereto and allowed to react therewith in an incubator for 2 hours under the conditions of 5% CO₂ and 37° C., followed by mild shaking for 1 minute. Then, the absorbance at 450 nm was measured.

The results of the indirect co-culture performed as described above could demonstrate that the proliferation of the human adipose tissue-derived mesenchymal stem cells in the test group was not inhibited compared to that in control group 1. Such results indicate that melanoma cells do not inhibit the proliferation of the human adipose tissue-derived mesenchymal stem cells (see FIGS. 7 and 8).

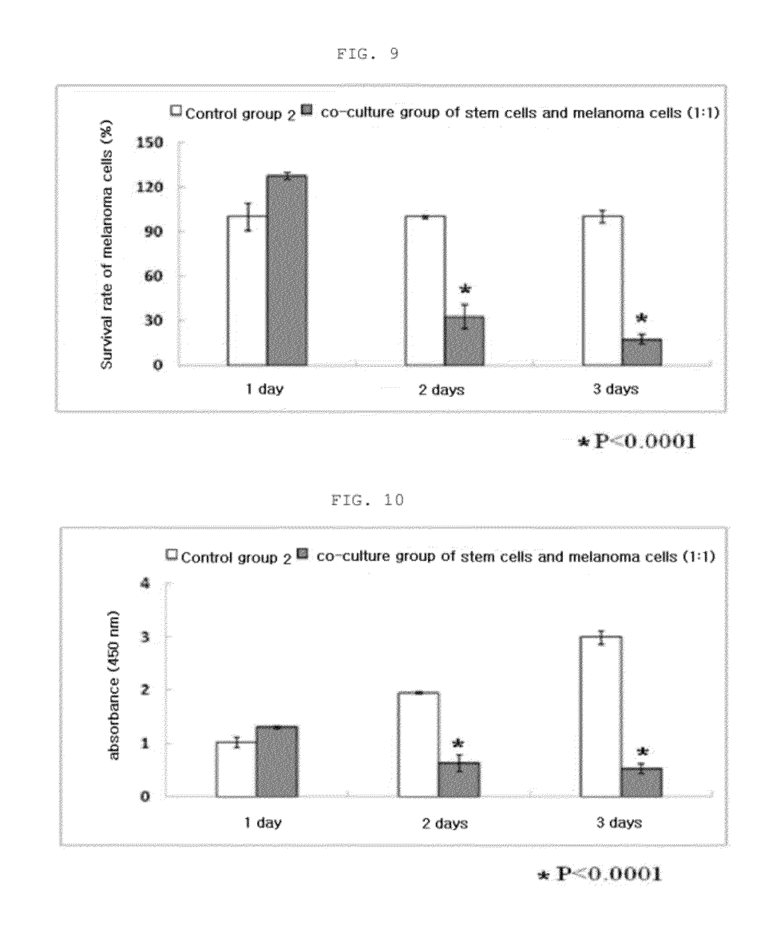

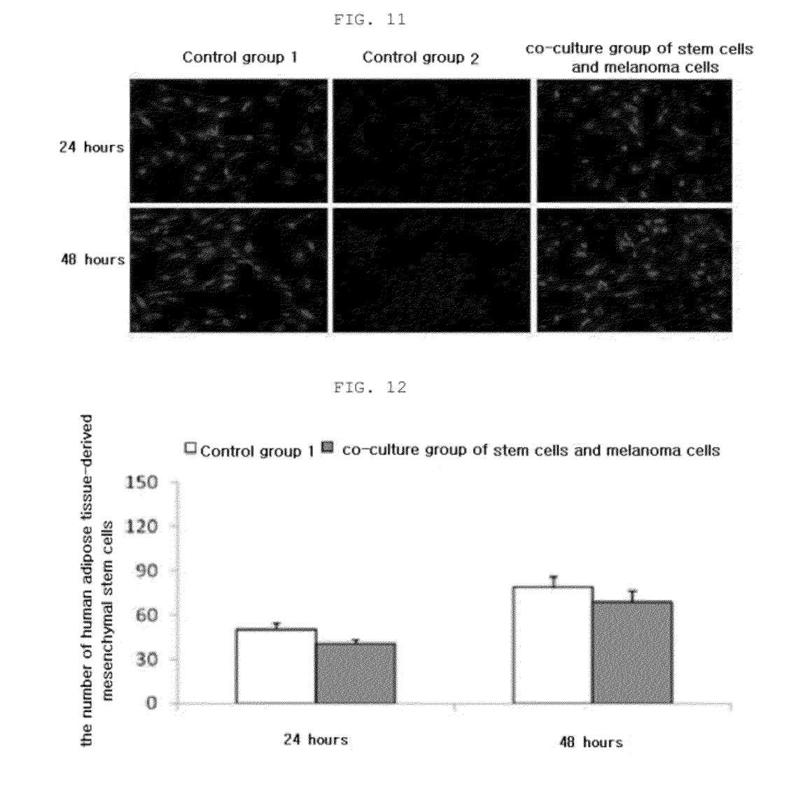

On the other hand, it could be seen that the proliferation of the melanoma cells in the test group was significantly inhibited compared to that in control group 2. Such results indicate that the human adipose tissue-derived mesenchymal stem cells inhibited the proliferation of the melanoma cells (see FIGS. 9 and 10).

### Example 6

### Examination of the Inhibitory Effect of Human Adipose Tissue-Derived Mesenchymal Stem Cells on Proliferation of Cancer Cells by Direct Co-Culture

In order to perform direct co-culture, in a test group, a 13-mm cell culture disk was placed in a 24-well plate, and ml of an antibiotic-containing OPTI-MEM medium (supplemented with 10% FBS, Gibco) was added thereto. Then, 5×10⁴ human adipose tissue-derived mesenchymal stem cells labeled with CM-DiI were added to the disk together with 5×10⁴ B16 melanoma cells and attached to the disk. Then, the cells were cultured under the conditions of 5% CO₂ and 37° C. for 1 and 2 days. In control group 1, the adipose tissue-derived mesenchymal stem cells (AT-MSCs) alone were cultured under the above conditions, and in control group 2, melanoma cells (B16) alone were cultured under the above conditions.

The disk having the cells attached thereto was washed with PBS and then stained with Hoechst 33342 solution for nuclear staining at 37° C. for 30 minutes. In order to count the cell number, the disk was mounted on a cover slide, and then photographed with a confocal laser scanning microscope, and the cell number was counted (see FIG. 11).

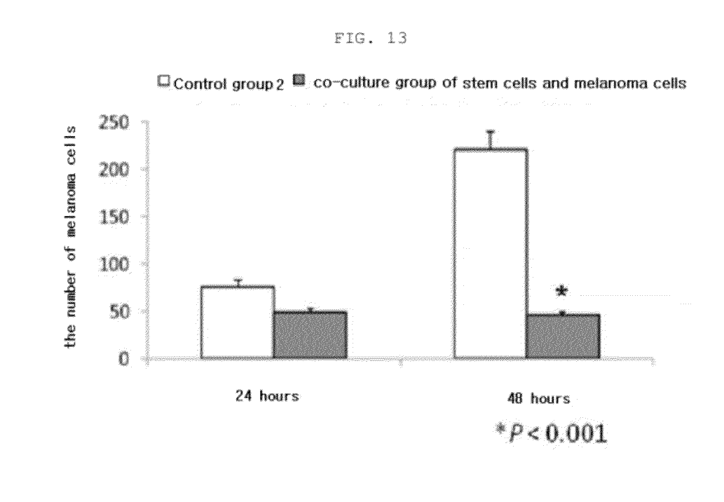

As can be seen in FIGS. 11 to 13, the results of the direct co-culture performed as described above indicated that the proliferation of the melanoma cells in the test group was significantly inhibited compared to that in the control group. Such results suggest that the proliferation of the melanoma cells was inhibited by the human adipose tissue-derived mesenchymal stem cells.

Therefore, the results of the in vivo and in vitro experiments performed in the present invention showed that the human adipose tissue-derived mesenchymal stem cells have an anticancer effect against melanoma and that the use of the human adipose tissue-derived mesenchymal stem cells provides a new anticancer therapy against melanoma.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

The present invention provides a new therapy method of preventing or treating cancer by using adult stem cells to activate the immune system. Because the adult stem cells of the present invention are used as a cell therapeutic agent, these cells are safer than existing chemotherapy. In addition, the adult stem cells of the present invention exhibit the effect of preventing or treating cancer by activating the immune system using a simple administration method such as intravenous injection. Thus, the adult stem cells of the present invention will be highly useful as a cell therapeutic agent for anticancer therapy.

1. A composition for preventing or treating cancer, wherein the composition contains as an active ingredient one or more selected from the group consisting of human adult stem cells and their secretory products.

2. The composition of claim 1, wherein the secretory products include one or more selected from the group consisting of cytokines, amino acids, and growth factors.

3. The composition of claim 1, wherein the secretory products include one or more selected from the group consisting of TGF (Transforming Growth Factor), bFGF (basic Fibroblast Growth Factor), IGF (Insulin-like Growth Factor), KGF (Keratinocyte Growth Factor), HGF (Hepatocyte Growth Factor), fibronectin, VEGF (Vascular Endothelial Growth Factor), and procollagen.

4. The composition of claim 1, wherein the adult stem cells are derived from tissue selected from the group consisting of including adipose, uterus, marrow, muscle, placenta, umbilical cord blood, hair follicle, and skin.

5. The composition of claim 4, wherein the adult stem cells are derived from adipose tissue.

6. The composition of claim 5, wherein the adult stem cells are human adipose tissue-derived mesenchymal stem cells (AT-MSCs).

7. The composition of claim 1, wherein the adult stem cells is contained in the composition at a concentration of 1×10⁵ cells/100 μl to 3×10⁵ cells/100 μl.

8. The composition of claim 1, wherein the composition for preventing or treating cancer is administered intravenously.

9. The composition of claim 1, wherein the composition for preventing or treating cancer is administered at the early onset stage of cancer diseases.

10. The composition of claim 1, wherein the cancer diseases is glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, meduliary carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcoma, chondrosarcoma, osteogenic sarcoma, or leukemia.

11. A composition for activating the immune system, wherein the composition contains as an active ingredient one or more selected from the group consisting of human adult stem cells and their secretory products.

12. The composition of claim 11, wherein the secretory products include one or more selected from the group consisting of cytokines, amino acids, and growth factors.

13. The composition of claim 11, wherein the secretory products include one or more selected from the group consisting of TGF, bFGF, IGF, KGF, HGF, fibronectin, VEGF, and procollagen.

14. The composition of claim 11, wherein the adult stem cells are derived from tissue selected from the group consisting of including adipose, uterus, marrow, muscle, placenta, umbilical cord blood, hair follicle, and skin.

15. The composition of claim 14, wherein the adult stem cells are derived from adipose tissue.

16. The composition of claim 15, wherein the adult stem cells are human adipose tissue-derived mesenchymal stem cells (AT-MSCs).

17. The composition of claim 11, wherein the adult stem cells is contained in the composition at a concentration of 1×10⁵ cells/100 μl to 3×10⁵ cells/100 μl.

18. The composition of claim 11, wherein the composition for activating the immune system is administered intravenously.

19. A method for preventing or treating cancer, which comprises a step of administering a composition containing as an active ingredient one or more selected from the group consisting of human adult stem cells and their secretory products.

20. The method of claim 19, wherein the secretory products include one or more selected from the group consisting of cytokines, amino acids, and growth factors.

21. The method of claim 19, wherein the secretory products include one or more selected from the group consisting of TGF, bFGF, IGF, KGF, HGF, fibronectin, VEGF, and procollagen.

22. The method of claim 19, wherein the step of administering is intravenous administration.

23. A method for activating the immune system, which comprises a step of administering a composition containing as an active ingredient one or more selected from the group consisting of human adult stem cells and their secretory products.

24. The method of claim 23, wherein the secretory products include one or more selected from the group consisting of cytokines, amino acids, and growth factors.

25. The method of claim 23, wherein the secretory products include one or more selected from the group consisting of TGF, bFGF, IGF, KGF, HGF, fibronectin, VEGF, and procollagen.

26. The method of claim 23, wherein the step of administering is intravenous administration.
